(19) United States
(12) Patent Application Publication
(10) Pub. No.: US 2018/0296815 A1
(43) Pub. Date: Oct. 18, 2018

(54) TOPICAL MEDICATION FOR SCALP

(71) Applicant: Carlos K. Wesley, New York, NY (US)

(72) Inventor: Carlos K. Wesley, New York, NY (US)

(21) Appl. No.: 15/949,320

(22) Filed: Apr. 10, 2018

Related U.S. Application Data
(60) Provisional application No. 62/485,513, filed on Apr. 14, 2017.

(51) Int. Cl. A61M 35/00; A61M 11/02; A61K 8/02; A61K 8/04; A61Q 7/00

(52) U.S. Cl. CPC A61M 35/003; A61M 11/02; A61K 8/027; A61K 8/046; A61K 2800/87; A61K 2800/26; A61K 2800/43; A61K 2800/81; A61Q 7/00

(57) ABSTRACT

A composition for treating a hair-growing surface from which hair follicles extend, the composition comprising an opaque material having sufficient shape and stability for covering the hair-growing surface, the opaque material including a medicament for promoting hair growth.

### CROSS-REFERENCE TO RELATED APPLICATIONS

This application claims the benefit of U.S. Provisional Application No. 62/485,513, filed Apr. 14, 2017, the content of which is incorporated herein by reference.

### BACKGROUND

This invention relates to topical medications for the scalp.

As hair follicles continue to cycle atop our head throughout our lifetime, they continue to grow back in slightly thinner caliber, the number of hairs per follicle decreases, and the follicle growth cycle gradually shortens. Thus, the overall hair coverage atop the head of both men and women decreases throughout their lifetime. As scalp hair coverage decreases, the scalp can be more easily seen. This is often an embarrassing physical feature that both men and women commonly try to either conceal the scalp or medically treat the scalp.

Medical treatments for decreased scalp coverage include FDA approved topical medication, Minoxidil, as well as various other topical components. Other components include topical finasteride for men and topical spironolactone, 17-alpha estradiol and melatonin for women. Shampoos for hair thickening may also include caffeine as a topical component.

### SUMMARY

In a general aspect of the invention, a composition for treating a hair-growing surface includes an opaque material having sufficient shape and stability for covering the hair-growing surface, the opaque material including a medicament for promoting hair growth.

Embodiments of these aspects of the invention may include one or more of the following features.

The opaque material is tinted to substantially match the color of the hair follicles within a hair-growing surface. The opaque material includes sprinkle-on fibers.

In certain embodiments, the opaque material further includes microcapsules. The opaque material includes foam or a spray. The opaque material can be in the form of a temporary tattoo.

In certain embodiments, the medicament is configured to be dissolved after a predetermined time or dissolved via interaction with intrinsic or extrinsic triggers. For example, the medicament is configured to be dissolved by the intrinsic fluids of the scalp. In other embodiments, the medicament is configured to be dissolved by extrinsic forces such as heat, a chemical, or when exposed to ultraviolet light.

In another aspect of the invention, a method of making a composition including combining an opaque material having sufficient shape and stability for covering a hair-growing surface, from which hair follicles extend, with a medicament for promoting hair growth.

In this aspect of the invention, tinting the composition to substantially match the color of the hair follicles within a hair-growing surface.

Embodiments of this aspect of the invention may include one or more of the following steps. The composition is provided as one or more of sprinkle-on fibers, microcapsules, a foam or a temporary tattoo.

In certain embodiments, the composition is dissolved after a predetermined period of time, or the composition may be dissolved by the intrinsic fluids of the scalp, or by extrinsic forces such as heat (e.g., hair dryer) or through the application of ultraviolet heat.

In another general aspect, an applicator for applying a composition to a hair-growing surface includes an applicator body. The applicator body includes a first reservoir configured to receive a first part of the composition, an application surface including a first opening through which the first part of the composition is applied to the hair-growing surface, a first channel extending from the first reservoir to the first opening, and a number of bristles extending from the application surface.

Aspects may include one or more of the following features.

The applicator body may include a second reservoir configured to receive a second part of the composition, a number of second openings disposed on the number of bristles through which the second part of the composition is applied to the hair-growing surface; and a number of second channels extending from the second reservoir, through the number of bristles, and to the number of second openings. The applicator may further include a first dispensing mechanism for dispensing the first part of the composition from the first opening and a second dispensing mechanism for dispensing the second part of the of the composition from the number of second openings. The first part of the composition may include a number of opaque fibers and the first dispensing mechanism may include a spreading mechanism for spreading the number of opaque fibers onto the hair-growing surface.

The second part of the composition may include a fluid medicament and the second dispensing mechanism may include a pumping mechanism for dispensing the fluid medicament from the number of second openings. The second part of the composition may include an aerosolized medicament and the second dispensing mechanism may be configured to cause aerosolization of a liquid form of the medicament from the number of second openings. The applicator may include a first control element for operating the first dispensing mechanism and a second control element for operating the second dispensing mechanism. The first control element and the second control element may be the same element.

The applicator may include a laser configured to emit light from the application surface onto the hair-growing surface. The first reservoir may be configured to receive a second part of the composition, the second part of the composition being applied to the hair-growing surface through the first opening.

Other features and advantages of the invention are apparent from the following description, and from the claims.

### DESCRIPTION OF DRAWINGS

FIG. 1 is a representative view of a portion of the scalp including a composition for treating a hair-growing surface in the form of sprinkle-on fibers.

FIG. 2 is a representative view of a portion of the scalp including another embodiment of a composition for treating a hair-growing surface in the form of microcapsules.

FIGS. 3A and 3B are cross-sectional representative views of the composition of FIG. 2 immediately after application and after a predetermined time period, respectively.

FIG. 4A is a cross-sectional representative view of a composition similar to FIG. 2 immediately after application.

FIG. 4B is a cross-sectional representative view of a composition similar to FIG. 2 during application of heat.

FIG. 4C is a cross-sectional representative view of a composition similar to FIG. 2 during application of ultraviolet light.

FIG. 4D is a cross-sectional representative view of a composition similar to FIG. 2 after application of heat or ultraviolet light.

FIG. 5 is a representative view of a portion of the scalp including a composition for treating a hair-growing surface in the form of a temporary tattoo.

FIG. 6A is a perspective view of an applicator for applying compositions including those shown in FIGS. 1-5.

FIG. 6B is a perspective view of the applicator shown in 6A such that its bottom is visible.

FIG. 6C is a cross-sectional view of the applicator shown in 6A.

FIG. 7 is an alternative embodiment of a medicament application device.

### DESCRIPTION

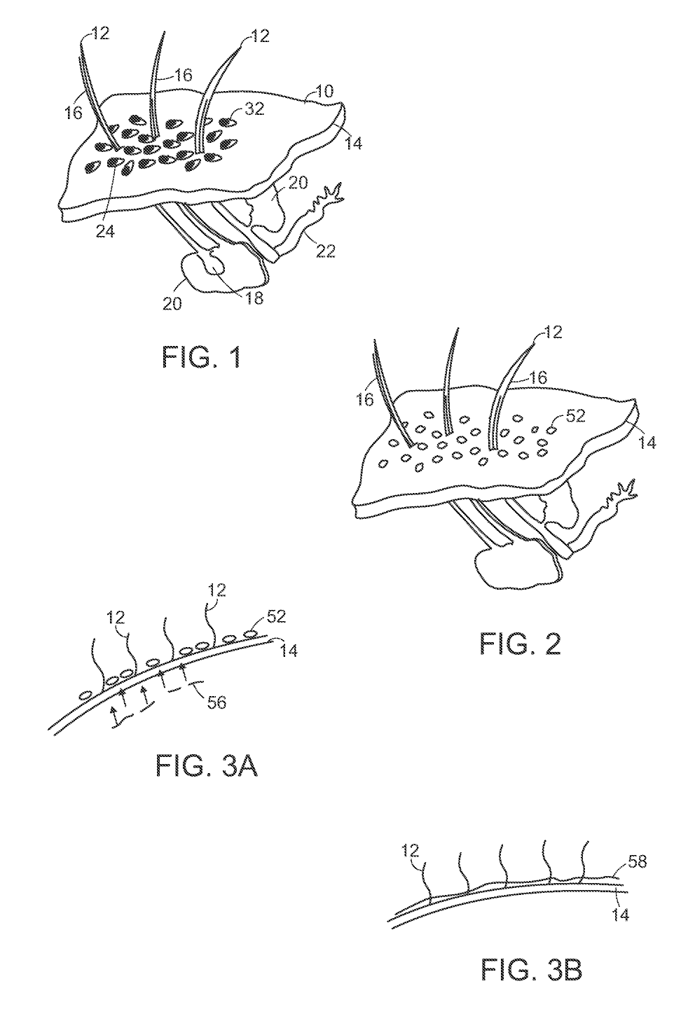

Referring to FIG. 1, a portion of a human's scalp 10 includes hairs 12, each hair extending through a layer of skin 14 and having a shaft 16 extending through the skin to a bulb 18. Shaft 16 of each hair 12 is attached to a sebaceous gland 20 and an arrector pili muscle 22. Hairs 12 are scarce leaving space 24 between adjacent hairs thereby the scalp. As the hairs become ever scarcer, the human scalp is said to be experiencing hair loss or baldness.

In the example of the human scalp 10 shown in in FIG. 1, exposed spaces 24 of the scalp between hairs 12 are covered by a composition in the form of sprinkle-on fibers 32. Sprinkle-on fibers 32 is formed of a non-medicated scalp concealing material having sufficient structure for carrying a medicament that promotes hair growth. For example, sprinkle-on fibers 32 may be formed of a keratin material. Sprinkle-on fibers 32 are opaque and preferably include a preselected tint that closely matches the color or tint of hairs 12 or shafts 16. Thus, sprinkle-on fibers are dual-purposed in that they both cover the exposed spaces of the scalp thereby covering the baldness while also administering medicament to scalp 10. Medicament, for example, includes one or more of the following ingredients:
0.1% Finasteride
6% Minoxidile
0.0125% Retinol
0.1% Caffeine.

The above ingredients in the given percentages are provided for a male hair growth topical blend.

For a female hair growth topical blend might include the following ingredients:
1% Spironolactone
0.025% 17-alpha-estradiol
0.1% Melatonin
5% Minoxidil
0.025% Retinol

While sprinkle-on fibers 32 persist on the scalp concealing any hair loss until washed off the scalp. Over time (e.g. 1-12 hours), the active ingredients in medicament will be absorbed into scalp 10 to promote hair growth. In one embodiment, the active ingredients are absorbed in about 2 hours.

Referring to FIG. 2, the composition is in the form of microcapsules 52. Like sprinkle-on fibers 42, microcapsules are opaque, preferably include a preselected tint that closely matches the color or tint of hairs 12 and include the medicament. In this embodiment, microcapsules 52 are formed of a dissolvable material that over time breaks down or disintegrates leaving behind the medicament on the surface of scalp 10 where it can be absorbed.

Referring to FIGS. 3A and 3B, microcapsules 52 are formed of a material such that when exposed to intrinsic fluids 56 of the scalp (sebum, sweat, etc.), the microcapsules break down and dissolve into a layer of dissolved medicament 58.

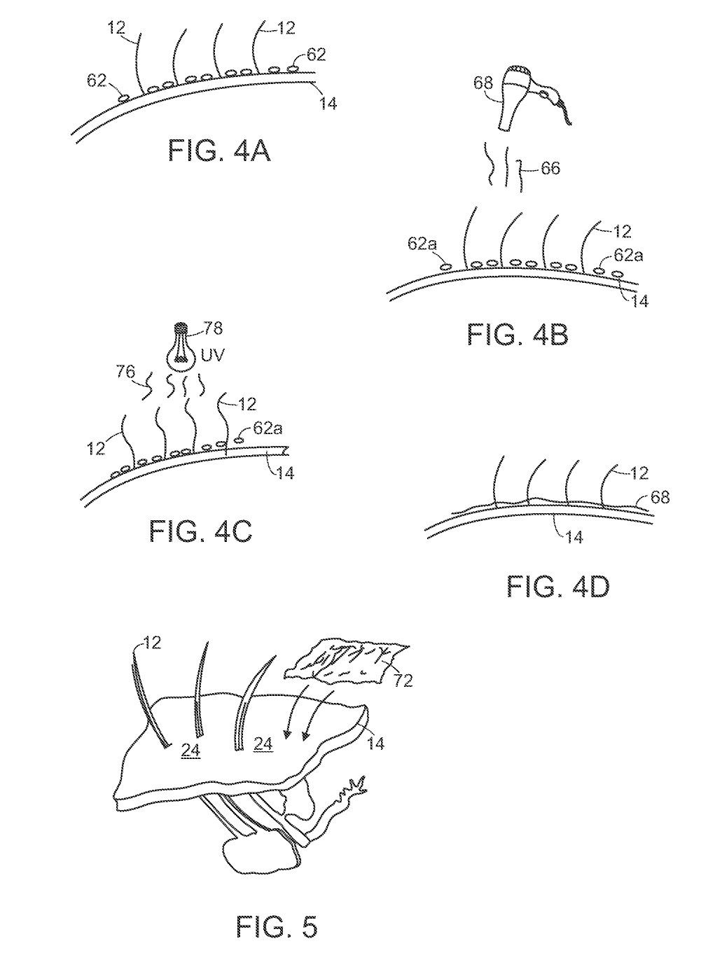

There are other mechanisms for dissolving the microcapsules. For example, referring to FIGS. 4A and 4B, microcapsules 62 are formed of a material such that when exposed to heat 66 (e.g., hair dryer 68), the microcapsules begin to break down and assume a less solid, absorbable form 62a.

Referring to FIG. 4C, in another embodiment, the microcapsules are formed of a material such that when exposed to certain frequencies or wavelengths of light 76 (e.g., ultraviolet lamp 78), the microcapsules break down and assume the less solid, absorbable form 62a.

Referring to FIG. 4D, regardless of the mechanisms for dissolving the microcapsules, a layer of dissolved medicament 68 is left behind that not only covers the exposed spaces of the scalp but also administers the desired medicament to scalp 10.

It is also important to appreciate that the medicament can be applied in other forms including sprays, foams, films, hair dyes, or shampoos. The medicament can be applied manually as a salve, as an aerosol, or with a pen or brush.

Referring to FIG. 5, the medicament is shown being applied as a temporary tattoo 72. The tattoo 72 generally has a color to match the color of the surrounding hair and over time will dissolve leaving behind the medicament.

In the composition described above in conjunction with FIGS. 1-5, the medicament was integrated as part of the concealing sprinkle-on fibers 32. In other embodiments, the concealing medicament is supplemented by additional concealing fibers that are kept separate from the concealing medicament. The concealing medicament is applied along the scalp and hair roots to medicate the patient's scalp, while providing a color or tint which matches the color of the patient's hair. The concealing fibers possess a texture that is more similar to that of hair than the texture of the medicament. For this reason, the concealing fibers should be applied higher up along the hair shafts on the patient's head (as opposed to the medicament which is placed on the scalp) with the goal of providing a more realistic hair texture than could be achieved by the concealing medicament alone. For example, the medicament and fibers are deposited independently and may be deposited sequentially or at the same time.

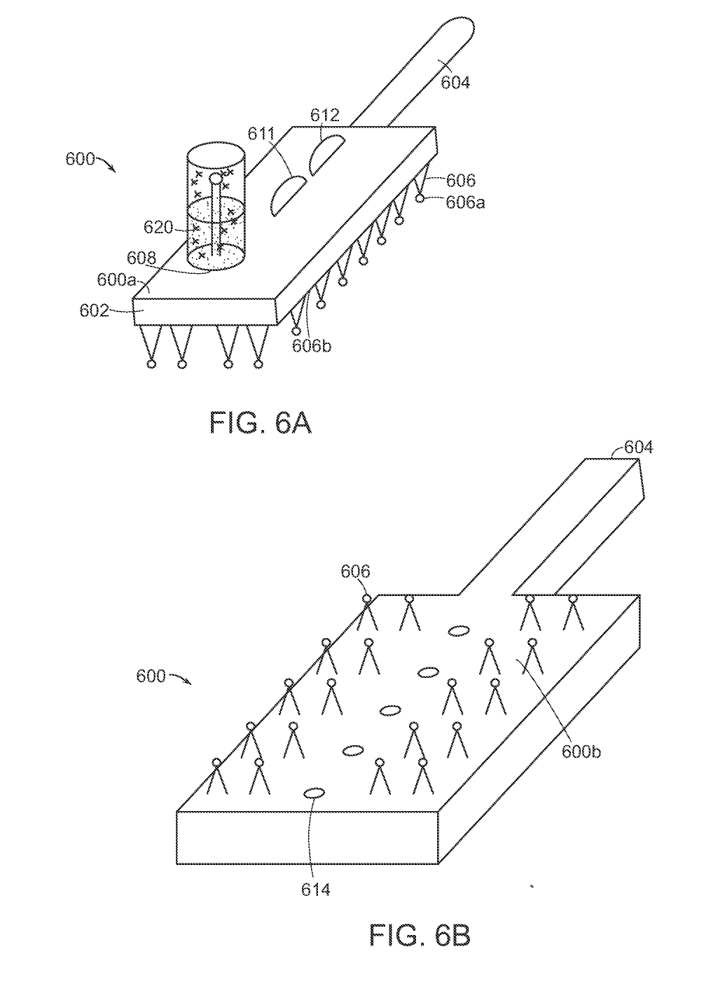

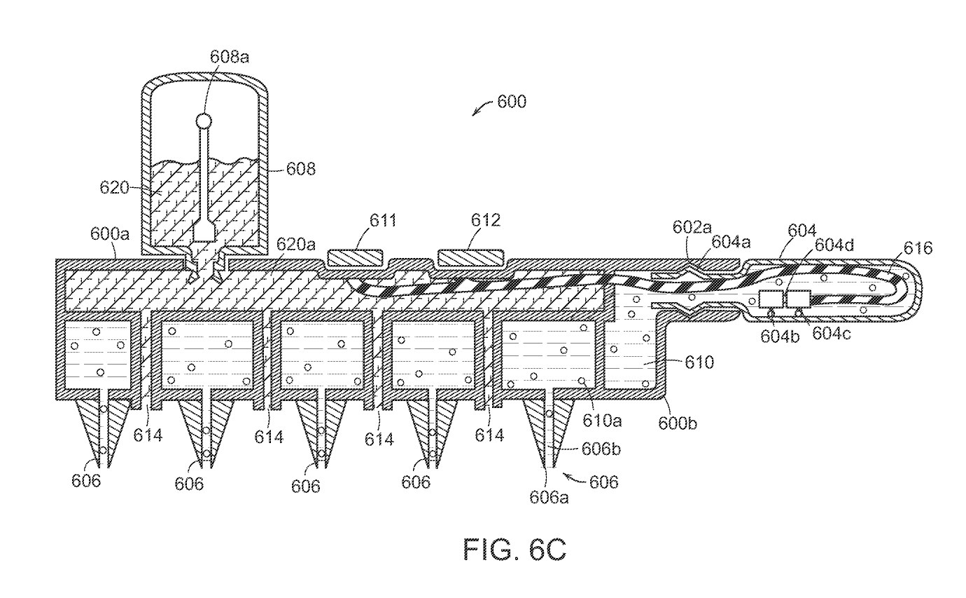

Referring now to FIGS. 6A-6C, an applicator 600 for applying a concealing medicament material 610 and concealing fibers 620 separately is shown. In this embodiment the applicator 600 includes an elongate rectangular housing (or body) 602 with a handle 604 positioned at one end of the housing 602. In some embodiments, the handle 604 may be configured to receive a replaceable cartridge for storing the concealing medicament material 610. The applicator 600 has bristles 606 disposed on a bottom face 600b of the housing 602. Each of the bristles 606 is shown to be conical in shape and includes a hollow interior (e.g., a channel) for receiving the concealing medicament material 610 from the cartridge before being applied through an opening at the tips 606a of the bristles to the patient scalp 10. The conical shape of the bristles is advantageous in separating any strands of the hair to provide clear access to the patient's scalp.

The housing 602 includes a fiber reservoir 608, a medicament control button 611, and a fiber control button 612 disposed on a top face 600a. The fiber reservoir 608 serves as a storage for the concealing fibers 620 prior to their application to the patient's scalp 10. In some embodiments, the applicator 600 includes a spreader (e.g., a broadcast spreader) for spreading the concealing fibers 620 as they leave the reservoir via a fiber delivery opening 614. The medicament control button 611 and the fiber control button 612, upon being pressed, are used to eject the concealing medicament material 610 and concealing fibers 620 respectively. Providing a separate medicament control button 611 and the fiber control button 612 allows for independent control of both active medicament material and concealing fiber by the user of the applicator. In some examples, the applicator 600 includes one or more pumps, spreading mechanisms or other distribution mechanisms (not shown) suitable for distributing the medicament and/or the concealing fibers from their respective reservoirs. In some examples, the one or more distribution mechanisms are controlled in an on/off manner by the medicament control button 611 and the fiber control button 612.

Referring to FIG. 6B, fiber delivery openings 614 are shown to be disposed on the bottom face 600b of applicator 600. The fiber delivery openings 614 are shown as a row of individual colinear fiber delivery openings running through the center of housing 502 and parallel to the handle 604 of the applicator 600. The fiber delivery openings 614 serve to release the concealing fiber 620 onto the patient's scalp 10. In alternative embodiments, an array (e.g., multiple rows) of fiber delivery openings can be used to dispense the concealing fibers 620.

Referring to FIG. 6C, the concealing medicament material 610 is shown to be contained within one or more internal medicament reservoirs 610a, and concealing fibers 620 are shown to be contained in an internal fiber reservoir 620a. The internal medicament reservoirs 610a are fluidly connected to openings 606a at the distal end of bristles 606 by a channel. Similarly, the fiber delivery openings 614 are each connected to internal fiber reservoir 620a by a channel. In some examples, the concealing medicament material 610 is not released from an internal medicament reservoir 610a and through openings 606b until control button 611 is depressed. The medicament control button 611 is connected to a power source 604c and controller 604b via electrical wiring 616 for powering and controlling the distribution mechanism (e.g., pump) tasked with dispensing the medicament material 610 from the applicator 600. Although in this embodiment, the power source 604c and the controller 604b are shown to be integrated with the handle 604, in other applications, they may be external to the applicator unit and connected via an external wire.

Referring further to FIG. 6C, the fiber reservoir 608 is shown to be connected via an opening to the internal fiber reservoir 620a. The internal fiber reservoir 620a contains concealing fibers 620 and, in some examples, does not allow the fibers to exit the internal reservoir unless instructed to do so. The fiber control button 612 functions such that when it is pressed, it allows for the concealing fibers 620 to be released from the internal fiber reservoir 620a and travel through the fiber delivery openings 614 to the target site on the patient's scalp. In some examples, the fiber control button 612 also activates a distribution mechanism such as a fiber spreader. The fiber control button 612 is connected to controller 604b via electrical wiring 616.

In some embodiments, fiber reservoir 608 may aerosolize the fibers upon leaving the reservoir. As illustrated in FIG. 6C, fiber reservoir 608 includes a fiber aerosolizer 608a. In an embodiment in which the concealment fibers 620 are aerosolized in this manner, it may be necessary to modify the control signal produced by controller 604b upon the pressing of fiber control button 612 to operate the fiber aerosolizer 608a.

The handle 604 includes an internal storage of active medicament material 610. In addition, the handle 604 includes an embossed section 604a near its opening so that the handle 604 can be locked securely in place within a locking portion 602a of the housing 602 during operation of the applicator 600. As mentioned above, the handle 604 contains the power source 604b and the controller 604c, which are electrically coupled via a wire 604d.

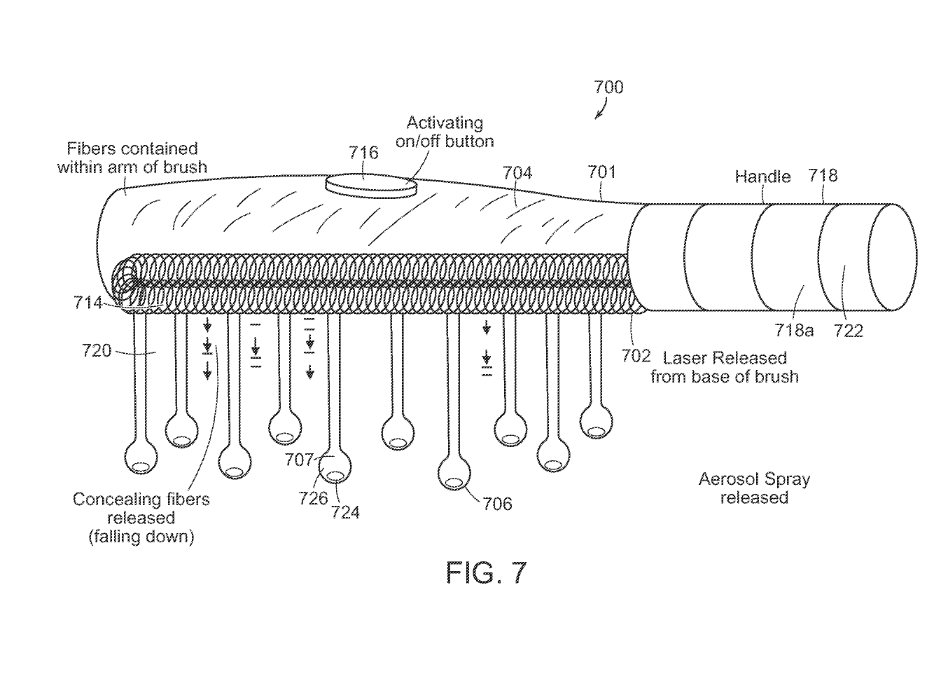

Referring to FIG. 7, an alternative embodiment of an applicator for use with delivering concealing fibers 720 that incorporate active medicament as described above in conjunction with FIGS. 1-5. As one can appreciate, use of an applicator can provide better control of the concealing fibers than through manual sprinkling.

In this embodiment of an applicator 700, bristles 706 have rounded tips at their distal end. Unlike the applicator 600 shown in FIGS. 6A-6C, bristles 706 are not only used to deliver medicament; in addition, they serve to separate strands of hair and provide better access for delivering concealing fibers 720 to the patient's scalp. In this embodiment, bristles 706 are of the type having ball-tips 707. The ball-tips 707 help bristles 706 glide through the hair more easily and prevent abrasions to the scalp, breaking or pulling of the hair. Applicator 700 includes fiber delivery openings 714, which are configured similarly to the fiber delivery openings 614 in applicator 600. That is, fiber delivery openings 714 are positioned in an array on surface 702 and between bristles 706.

The applicator 700 has a reservoir 704 inside its base 701. This reservoir 704 and base 701 function similarly to the reservoirs 620a and housing 602 (see FIG. 6C), respectively.

Further referring to FIG. 7, the applicator 700 includes a power button 716. The power button 716 functions such that when pressed, concealing fibers 720 are ejected from the applicator 700 on to a target site on the patient's scalp. Applicator 700 also includes a handle 718, which has an internal storage 722 of aerosol medicament spray 718a. Upon activation of power button 716, aerosol spray 718a is also forced out of storage 722 in the handle 718 and through the openings 724 on the tips 726 of the bristles 706.

In the embodiment shown in FIG. 7, the applicator includes a laser 702 for providing low level light to stimulate hair growth (also referred to as “photo stimulation”). Laser 702 may be controlled to provide light during the application of the concealing fibers 720 or after they have been placed on the scalp. Photo stimulation can promote hair growth by using light wavelengths of 614-624 nm, 668-684 nm, 751-772 nm, and 813-846 nm. Applicator 700 may include an on/off switch (not shown) for turning the laser 702 on and off. In some embodiments, a single on/off button may be used to initiate the application of the sprinkle-on fibers as well as the laser 702.

Other embodiments are within the scope of the following claims.

What is claimed is:

1. An applicator for applying a composition to a hair-growing surface, the applicator comprising:
an applicator body comprising,
a first reservoir configured to receive a first part of the composition,
an application surface including a first opening through which the first part of the composition is applied to the hair-growing surface,
a first channel extending from the first reservoir to the first opening, and
a plurality of bristles extending from the application surface.

2. The applicator of claim 1 wherein the applicator body further comprises:
a second reservoir configured to receive a second part of the composition,
a plurality of second openings disposed on the plurality of bristles through which the second part of the composition is applied to the hair-growing surface;
a plurality of second channels extending from the second reservoir, through the plurality of bristles, and to the plurality of second openings.

3. The applicator of claim 2 wherein the applicator further comprises a first dispensing mechanism for dispensing the first part of the composition from the first opening and a second dispensing mechanism for dispensing the second part of the composition from the plurality of second openings.

4. The applicator of claim 3 wherein the first part of the composition comprises a plurality of opaque fibers and the first dispensing mechanism comprises a spreading mechanism for spreading the plurality of opaque fibers onto the hair-growing surface.

5. The applicator of claim 4 wherein the second part of the composition comprises a fluid medicament and the second dispensing mechanism comprises a pumping mechanism for dispensing the fluid medicament from the plurality of second openings.

6. The applicator of claim 4 wherein the second part of the composition comprises an aerosolized medicament and the second dispensing mechanism is configured to cause aerosolization of a liquid form of the medicament from the plurality of second openings.

7. The applicator of claim 3 further comprising a first control element for operating the first dispensing mechanism and a second control element for operating the second dispensing mechanism.

8. The applicator of claim 7 wherein the first control element and the second control element are the same element.

9. The applicator of claim 1 further comprising a laser configured to emit light from the application surface onto the hair-growing surface.

10. The applicator of claim 1 wherein the first reservoir is further configured to receive a second part of the composition, the second part of the composition being applied to the hair-growing surface through the first opening.

11. A composition for treating a hair-growing surface from which hair follicles extend, the composition comprising an opaque material having sufficient shape and stability for covering the hair-growing surface, the opaque material including a medicament for promoting hair growth.

12. The composition of claim 11 wherein the opaque material is tinted to substantially match the color of hair follicles.

13. The composition of claim 11 wherein the opaque material comprises sprinkle-on fibers.

14. The composition of claim 11 wherein the medicament is configured to be dissolved by the intrinsic fluids of the scalp.

15. The composition of claim 11 wherein the medicament is configured to be dissolved by heat.

16. A method of making a composition comprising combining an opaque material having sufficient shape and stability for covering a hair-growing surface, from which hair follicles extend, with a medicament for promoting hair growth.

17. The method of claim 16 further comprising tinting the composition to substantially match the color of the hair follicles.

18. The method of claim 16 further comprising providing the composition as sprinkle-on fibers.

19. The method of claim 16 further comprising providing the composition to be dissolved by the intrinsic fluids of the scalp.

20. The method of claim 16 further comprising providing the composition to be dissolved by heat.
